(19) United States
(12) Patent Application Publication
(10) Pub. No.: US 2007/0104779 A1
(43) Pub. Date: May 10, 2007

(54) Treatment with omega-3 fatty acids and products thereof

(76) Inventors: Roelof M.L. Rongen, Califon, NJ (US); Robert A. Shalwitz, Bexley, OH (US)

Correspondence Address: ARENT FOX PLLC, 1050 CONNECTICUT AVENUE, N.W., SUITE 400, WASHINGTON, DC, 20036, US

(21) Appl. No.: 11/267,581

(22) Filed: Nov. 7, 2005

(51) Int. Cl. A61K 9/64; A61K 31/22; A61K 31/202

(52) U.S. Cl. 424/456; 514/546; 514/560

(57) ABSTRACT

A method for reducing non-HDL cholesterol levels of a subject comprising administering a pharmaceutical composition comprising omega-3 fatty acids to the subject in an amount sufficient to reduce non-HDL cholesterol. The method may include a reduction of the triglyceride levels of the subject and, optionally, an increase in the HDL cholesterol levels of the subject. These methods can be used concurrently with hypertriglyceridemia therapy prescribed by a doctor.

### FIELD OF THE INVENTION

The present invention relates to a method utilizing a single administration or a unit dosage of omega-3 fatty acids for the treatment of subjects with hypertriglyceridemia, coronary heart disease (CHD), vascular disease, artheroscierotic disease and related conditions and the prevention or reduction of cardiovascular and vascular events.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into blood plasma. High levels of total cholesterol (total-C), LDL-C, and apolipoprotein B (a membrane complex for LDL-C and VLDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C.

Agents, such as omega-3 fatty acids, have been used to treat post-myocardial infarction (MI) and adult endogenous hyperlipidemias of hypercholesterolemias and of hypertriglyceridemias, which are generally categorized as “cardiovascular events”.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum LDL-cholesterol, increase serum HDL-cholesterol, lower serum triglycerides, lower systolic and diastolic blood pressure and the pulse rate, and lower the activity of the blood coagulation factor VII-phospholipid complex. Further, omega-3 fatty acids seem to be well tolerated, without giving rise to any severe side effects.

One form of omega-3 fatty acid is a concentrate of omega-3, long chain, polyunsaturated fatty acids from fish oil containing DHA and EPA and is sold under the trademark Omacor®. Such a form of omega-3 fatty acid is described, for example, in U.S. Pat. Nos. 5,502,077, 5,656,667 and 5,698,594, each incorporated herein by reference.

Previously studies have disclosed treatments of subjects with Omacor® omega-3 fatty acid compositions for the treatment and prevention of cardiovascular events. For example, it has been shown that subjects treated with 4 grams per day of Omacor® omega-3 acids over a 6-week period reduced TG, reduced VLDL-C, increased LDL-C and increased HDL-C in the serum of the treated subjects. Pownall et al., Atherosclerosis, 143: 285-297 (1999). A similar study showed that subjects treated with 4 grams per day of Omacor® omega-3 acids over a 4-month period reduced TG, reduced VLDL-C, increased LDL-C and increased HDL-C in the serum of the treated subjects. Harris et al., J. of Cardiovascular Risk, 243(4): 268-391 (1997). However, researchers are finding that non-HDL cholesterol is an important indicator of hypertriglyceridemia, vascular disease, artherosclerotic disease and related conditions. In fact, recently non-HDL cholesterol reduction has been specified as a treatment objective in NCEP ATP III.

### SUMMARY OF THE INVENTION

It has now been found that fatty acid compositions containing a concentration of omega-3 fatty acids, salts or derivatives thereof, have an advantageous effect on treatment of hypertriglyceridemia, vascular disease, artherosclerotic disease and related conditions. A concentration of omega-3 fatty acids has been found to reduce serum non-HDL cholesterol. Therefore, the present invention provides products and methods comprising administration of concentrated amounts of omega-3 fatty acids (e.g., the Omacor® omega-3 acids) in a unit dosage.

Some embodiments of the present invention provide an administration of a unit dosage of omega-3 fatty acids that can provide an effective pharmaceutical treatment of coronary heart disease, vascular disease, and related disorders, events, and/or symptoms.

Other embodiments of the present invention provide a method of reducing the risk or progression of cardiovascular diseases or risk factors thereof, including hypertriglyceridemia, vascular disease, artherosclerotic disease and related conditions, by administering omega-3 fatty acids.

Other embodiments of the present invention are directed to a product, for example, a unit dosage, of omega-3 fatty acids. In one aspect of the embodiment, the product is used in the treatment of hypertriglyceridemia, vascular disease, artherosclerotic disease and related conditions, the prevention or reduction of cardiovascular and vascular events.

Some embodiments of the present invention comprise methods for the reduction of non-HDL cholesterol comprising administration of a pharmaceutical composition containing omega-3 fatty acids to a subject in need of such treatment. For example, some embodiments of the present invention comprise methods for the reduction of non-HDL cholesterol comprising administration of a pharmaceutical composition containing omega-3 fatty acids to a subject in need of blood lipid regulation.

Some embodiments of the present invention comprise methods of reducing non-HDL cholesterol as an adjunct to hypertriglyceridemia therapy in a subject for which hypertriglyceridemia treatment is prescribed by a doctor, comprising administration of a pharmaceutical composition containing omega-3 fatty acids to the subject in an amount sufficient to reduce non-HDL cholesterol.

Another embodiment of the present invention comprises methods of reducing non-HDL cholesterol and triglyceride levels in a subject for which hypertriglyceridemia treatment is prescribed by a doctor, comprising administration of a pharmaceutical composition containing omega-3 fatty acids to the subject in an amount sufficient to reduce non-HDL cholesterol and triglyceride levels.

In preferred embodiments the pharmaceutical compositions comprise Omacor® omega-3 fatty acids, as described in U.S. Pat. Nos. 5,502,077, 5,656,667 and 5,698,594. In other preferred embodiments the pharmaceutical compositions comprise omega-3 fatty acids present in a concentration of at least 40% by weight as compared to the total fatty acid content of the composition.

In still other preferred embodiments the omega-3 fatty acids comprise at least 60% by weight of EPA and DHA as compared to the total fatty acid content of the composition, and the EPA and DHA are in a weight ratio of EPA:DHA of from 99:1 to 1:99, preferably from 1:2 to 2:1.

Another embodiment provides the use of omega-3 fatty acids for the preparation of a medicament useful for the reduction of non-HDL cholesterol levels in a subject.

Other features and advantages of the present invention will become apparent to those skilled in the art upon examination of the following or upon learning by practice of the invention.

### DESCRIPTION OF THE PREFERRED EMBODIMENTS

The present invention is directed to the utilization of omega-3 fatty acids, preferably concentrated omega-3 fatty acids, for the treatment of hypertriglyceridemia, mixed dyslipidemia, hypercholesterolemia, vascular disease, artherosclerotic disease and related conditions, the prevention or reduction of cardiovascular and vascular events, and the reduction of insulin resistance, fasting glucose levels and postprandial glucose levels, and a unit dosage comprising omega-3 fatty acids.

As used herein, the term “omega-3 fatty acids” includes natural or synthetic omega-3 fatty acids, or pharmaceutically acceptable esters, derivatives, conjugates (see, e.g., Zaloga et al., U.S. patent application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 95% by weight, more preferably about 25 to about 75% by weight, still more preferably about 40 to about 55% by weight, and most preferably about 46% by weight of EPA. Preferably the omega-3 fatty acids comprise about 5 to about 95% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated.

The EPA:DHA ratio may be from 99:1 to 1:99, preferably 4:1 to 1:4, more preferably 3:1 to 1:3, most preferably 2:1 to 1:2.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Omacor® omega-3 acid (K85EE, Pronova Biocare A.S., Lysaker, Norway) and preferably comprises the following characteristics (per dosage form):

| Test | Minimum Value | Maximum Value |
|---|---|---|
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n − 3 fatty acids | 90% (w/w) | |

The omega-3 fatty acids may be administered in a capsule, a tablet, a powder that can be dispersed in a beverage, or another solid oral dosage form, a liquid, a soft gel capsule or other convenient oral dosage form such as oral liquid in a capsule, as known in the art. In some embodiments, the capsule comprises a hard gelatin. The omega-3 fatty acids may also be contained in a liquid suitable for injection or infusion.

The active ingredients of the present invention comprise omega-3 fatty acids and may be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

In most embodiments, excipients primarily include surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, co-solvents, oils or combinations thereof is generally known in the pharmaceutical arts, and as would be understood to one skilled in the art, any suitable surfactant may be used in conjunction with the present invention and embodiments thereof.

The omega-3 fatty acids may be administered in a daily amount from about 0.1 g to about 10 g, more preferably about 1 g to about 6 g, and most preferably from about 2 g to about 4 g.

The daily dosages of omega-3 fatty acids may be administered in from 1 to 10 dosages, with the preferred number of dosages from 1 to 4 times a day, most preferred 1 to 2 times a day. The administration is preferably oral administration, although other forms of administrations that provide a unit dosage of omega-3 fatty acids may be used. The amount of omega-3 fatty acids may also be sufficient to reduce triglyceride levels of the subject, and/or increase HDL-C levels of the subject.

The present invention may be useful for subjects with baseline triglyceride levels greater than or equal to 500 mg/dL. The present invention may also be useful for subjects with baseline triglyceride levels between about 200 mg/dL and about 499 mg/dL.

The subject to be treated may be human, mammalian animal or bird, with human preferred. Preferably, the subject is in need of non-HDL cholesterol reduction. The subject may be in need of blood lipid regulation. The subject may be receiving hypertriglyceridemia therapy which is prescribed by a doctor.

### EXAMPLE 1

The effects of Omacor® omega-3 acids 4 grams per day have been assessed in two randomized, placebo-controlled double-blind, parallel-group studies of 84 adult subjects (i.e., 42 adult subjects on Omacor® and 42 adult subjects on placebo). Adult subjects with baseline triglyceride levels between 500 and 2000 mg/dL were enrolled in the studies for durations of 6 and 16-weeks. The median triglyceride and LDL-C levels in these subjects were 792 mg/dL and 100 mg/dL, respectively. The median HDL-C levels in these subjects were 23.0 mg/dL. The Omacor® was supplied as a liquid-filled gel capsule for oral administration. Each one gram capsule of Omacor® contained at least 900 mg of ethyl esters of omega-3 fatty acids, which comprises predominantly eicosapentaenoic acid (EPA) (about 465 mg) and docosahexaenoic acid (DHA) (about 375 mg).

The study showed that the administration of Omacor® at 4 grams per day reduced median TG, VLDL-C, and non-HDL-C levels and increased median HDL-C in the serum subjects treated with Omacor® relative to placebo, as shown in Table 1. Treatment of subjects with highly concentrated omega-3 fatty acids is useful in the treatment of subjects with hypertriglyceridemia, coronary heart disease (CHD), vascular disease, artherosclerotic disease and related conditions and the prevention or reduction of cardiovascular and vascular events.

TABLE 1
Median Baseline and Percent Change from Baseline in Lipid
Parameters in Subjects with Very High TG Levels (≧500 mg/dL)

| | TG | | LDL-C | | CHOL | | HDL-C | | VLDL-C | | non-HDL-C | |
|---|---|---|---|---|---|---|---|---|---|---|---|---|
| | BL | % Chg | BL | % Chg | BL | % Chg | BL | % Chg | BL | % Chg | BL | % Chg |
| Placebo | 788 | +6.7 | 108 | −4.8 | 314 | −1.7 | 24 | 0.0 | 175 | −0.9 | 292 | −3.6 |
| Omacor | 816 | −44.9 | 89 | +44.5 | 296 | −9.7 | 22 | +9.1 | 175 | −41.7 | 271 | −13.8 |
| 4 g/day | | | | | | | | | | | | |
| Difference | | −51.6 | | +49.3 | | −8.0 | | +9.1 | | −40.8 | | −10.2 |

| | | | | | | |
|---|---|---|---|---|---|---|
| P-value | <0.0001 | 0.0004 | 0.0047 | 0.0003 | <0.0001 | 0.0013 |
| Omacor vs | | | | | | |
| placebo | | | | | | |
| P-value | <0.0001 | <0.0001 | <0.0001 | <0.0001 | <0.0001 | <0.0001 |
| Omacor vs | | | | | | |
| baseline | | | | | | |

BL = Baseline (mg/dL):
% Chg = Percent Change from Baseline;
Difference = Omacor − Placebo

### EXAMPLE 2

The effect of 4 grams per day of Omacor® omega-3 fatty acids on the lipid parameters, i.e. triglyceride levels (TG), total cholesterol, high density lipoproteins (HDL), low density lipoproteins (LDL) and very low density lipoprotein (VLDL), of patients with different baseline TG levels has been evaluated. The Omacor® omega-3 fatty acids were supplied as a liquid-filled gel capsule for oral administration. Each one gram capsule of Omacor® contained at least 900 mg of ethyl esters of omega-3 fatty acids, which comprises predominantly eicosapentaenoic acid (EPA) (about 465 mg) and docosahexaenoic acid (DHA) (about 375 mg). As shown in Table 2, the effectiveness of Omacor® omega-3 fatty acids is dependent on the baseline TG levels of the treated of patients.

TABLE 2
Percent Change in Lipid Parameters in Patients after
administration of Omacor ®

| Baseline TG | | Total | | | | |
|---|---|---|---|---|---|---|
| (mg/dL) | TG | cholesterol | HDL | LDL | VLDL | Non-HDL |
| 0-199 | −22.5 | 3.5 | 5.2 | 10.7 | −31.6 | 3.8 |
| 200-299 | −23.0 | 0.2 | 7.3 | 5.9 | −21.2 | −0.5 |
| 300-399 | −26.1 | −1.1 | 6.1 | 9.9 | −22.3 | −1.2 |
| 400-499 | −25.9 | −4.7 | 12.6 | 18.9 | −8.8 | −7.3 |
| 500-599 | −39.8 | −4.8 | 9.8 | 44.7 | −34.9 | −6.2 |
| 600-699 | −36.9 | −3.6 | 8.1 | 47.6 | −25.6 | −5.0 |
| 700- | −39.9 | −15.4 | 16.5 | 40.3 | −26.0 | −17.8 |

We claim:

1. A method for reducing non-HDL cholesterol levels of a subject comprising administering to the subject a pharmaceutical composition in an amount sufficient to reduce non-HDL cholesterol, the composition containing natural or synthetic omega-3 fatty acids, or pharmaceutically acceptable esters, derivatives, conjugates, precursors or salts thereof, or mixtures thereof, and one or more pharmaceutically acceptable excipients.

2. The method of claim 1, wherein the omega-3 fatty acids are contained in a liquid.

3. The method of claim 2, wherein the liquid is contained in a gelatin capsule.

4. The method of claim 1, wherein the omega-3 fatty acids are contained in a powder, a tablet, or another solid oral dosage form.

5. The method of claim 1, wherein the omega-3 fatty acids are contained in a liquid suitable for injection or infusion.

6. The method of claim 1, wherein the omega-3 fatty acids are present in a concentration of at least 40% by weight as compared to the total fatty acid content of the composition.

7. The method of claim 1, wherein the omega-3 fatty acids are present in a concentration of at least 50% by weight as compared to the total fatty acid content of the composition.

8. The method of claim 1, wherein the omega-3 fatty acids are present in a concentration of at least 60% by weight as compared to the total fatty acid content of the composition.

9. The method of claim 1, wherein the omega-3 fatty acids are present in a concentration of at least 70% by weight as compared to the total fatty acid content of the composition.

10. The method of claim 1, wherein the omega-3 fatty acids are present in a concentration of at least 80% by weight as compared to the total fatty acid content of the composition.

11. The method of claim 1, wherein the omega-3 fatty acids are present in a concentration of at least 90% by weight as compared to the total fatty acid content of the composition.

12. The method of claim 1, wherein the omega-3 fatty acids comprise at least 50% by weight of EPA and DHA as compared to the total fatty acid content of the composition.

13. The method of claim 1, wherein the omega-3 fatty acids comprise at least 60% by weight of EPA and DHA as compared to the total fatty acid content of the composition.

14. The method of claim 1, wherein the omega-3 fatty acids comprise at least 70% by weight of EPA and DHA as compared to the total fatty acid content of the composition.

15. The method of claim 1, wherein the omega-3 fatty acids comprise at least 80% by weight of EPA and DHA as compared to the total fatty acid content of the composition.

16. The method of claim 1, wherein the omega-3 fatty acids comprise about 84% by weight of EPA and DHA as compared to the total fatty acid content of the composition.

17. The method of claim 1, wherein the omega-3 fatty acids comprise about 5% to about 95% by weight of EPA as compared to the total fatty acid content of the composition.

18. The method of claim 1, wherein the omega-3 fatty acids comprise about 25% to about 75% by weight of EPA as compared to the total fatty acid content of the composition.

19. The method of claim 1, wherein the omega-3 fatty acids comprise about 40% to about 55% by weight of EPA as compared to the total fatty acid content of the composition.

20. The method of claim 1, wherein the omega-3 fatty acids comprise about 46% by weight of EPA as compared to the total fatty acid content of the composition.

21. The method of claim 1, wherein the omega-3 fatty acids comprise about 5% to about 95% by weight of DHA as compared to the total fatty acid content of the composition.

22. The method of claim 1, wherein the omega-3 fatty acids comprise about 25% to about 75% by weight of DHA as compared to the total fatty acid content of the composition.

23. The method of claim 1, wherein the omega-3 fatty acids comprise about 30% to about 60% by weight of DHA as compared to the total fatty acid content of the composition.

24. The method of claim 1, wherein the omega-3 fatty acids comprise about 38% by weight of DHA as compared to the total fatty acid content of the composition.

25. The method of claim 1, wherein the omega-3 fatty acids are the only active ingredient in the composition.

26. The method of claim 1, wherein omega-3 fatty acids comprise omega-3 polyunsaturated, long-chain fatty acids, esters of omega-3 fatty acids with glycerol, esters of omega-3 fatty acids and a primary, secondary or tertiary alcohol, or mixtures thereof.

27. The method of claim 26, wherein the omega-3 fatty acids comprise EPA, DHA, α-linolenic acid, or mixtures thereof.

28. The method of claim 1, wherein the omega-3 fatty acids comprise EPA and DHA in a ratio of EPA:DHA from 99:1 to 1:99.

29. The method of claim 1, wherein the omega-3 fatty acids comprise EPA and DHA in a ratio of EPA:DHA from 4:1 to 1:4.

30. The method of claim 1, wherein the omega-3 fatty acids comprise EPA and DHA in a ratio of EPA:DHA from 3:1 to 1:3.

31. The method of claim 1, wherein the omega-3 fatty acids comprise EPA and DHA in a ratio of EPA:DHA from 1:2 to 2:1.

32. The method of claim 1, wherein the composition further comprises one or more antioxidants, oils, lubricants, lecithin, or a mixture thereof.

33. The method of claim 1, wherein the omega-3 fatty acids are administered in an amount from about 0.1 g to about 10 g per day.

34. The method of claim 1, wherein the omega-3 fatty acids are administered in an amount from about 1 g to about 6 g per day.

35. The method of claim 1, wherein the omega-3 fatty acids are administered in an amount from about 2 g to about 4 g per day.

36. The method of claim 1, wherein the subject is a human, mammalian animal or bird.

37. The method of claim 1, wherein the subject is in need of non-HDL cholesterol reduction.

38. The method of claim 1, wherein the subject is in need of blood lipid regulation.

39. The method of claim 1, wherein the subject is receiving hypertriglyceridemia therapy which is prescribed by a doctor.

40. The method of claim 1, wherein the amount of omega-3 fatty acids is also sufficient to reduce triglyceride levels of the subject.

41. The method of claim 1, wherein the amount of omega-3 fatty acids is also sufficient to increase HDL-C levels of the subject.

42. The method of claim 1, wherein the composition is administered in 1 or 2 dosages per day.

43. The method of claim 1, wherein the composition is administered orally.

44. The method of claim 1, wherein the subject has a baseline triglyceride level of greater than or equal to 500 mg/dL.

45. The method of claim 1, wherein the subject has a baseline triglyceride level between 200 mg/dL to 499 mg/dL.

46. A method for reducing non-HDL cholesterol levels in a human subject, comprising administering to the subject a pharmaceutical composition in an amount sufficient to reduce non-HDL cholesterol, the composition containing natural or synthetic omega-3 fatty acids, or pharmaceutically acceptable esters, derivatives, conjugates, precursors or salts thereof, or mixtures thereof, and one or more pharmaceutically acceptable excipients, wherein the omega-3 fatty acids comprise at least 60% by weight of EPA and DHA as compared to the total fatty acid content of the composition, and wherein the EPA and DHA are in a weight ratio of EPA:DHA of from 99:1 to 1:99.

47. The method of claim 46, wherein the omega-3 fatty acids are present in a concentration of at least 70% by weight as compared to the total fatty acid content of the composition.

48. The method of claim 46, wherein the EPA and DHA are in a weight ratio of EPA:DHA of from 1:2 to 2:1.

49. The method of claim 46, wherein the subject has a baseline triglyceride level of greater than or equal to 500 mg/dL.

50. The method of claim 46, wherein the subject has a baseline triglyceride level between 200 mg/dL to 499 mg/dL.

51. A method for reducing non-HDL cholesterol levels and triglyceride levels of a subject for which hypertriglyceridemia treatment is prescribed by a doctor, comprising administering to the subject a pharmaceutical composition in an amount sufficient to reduce non-HDL cholesterol levels and triglyceride levels, the composition containing natural or synthetic omega-3 fatty acids, or pharmaceutically acceptable esters, derivatives, conjugates, precursors or salts thereof, or mixtures thereof, and one or more pharmaceutically acceptable excipients.

52. The method of claim 51, wherein the omega-3 fatty acids comprise at least 60% by weight of EPA and DHA as compared to the total fatty acid content of the composition, and wherein the EPA and DHA are in a weight ratio of EPA:DHA of from 99:1 to 1:99.

53. The method of claim 51, wherein the omega-3 fatty acids are present in a concentration of at least 70% by weight as compared to the total fatty acid content of the composition.

54. The method of claim 52, wherein the EPA and DHA are in a weight ratio of EPA:DHA of from 1:2 to 2:1.

55. The method of claim 51, wherein the subject has a baseline triglyceride level of greater than or equal to 500 mg/dL.

56. The method of claim 51, wherein the subject has a baseline triglyceride level between 200 mg/dL to 499 mg/dL.

57. A method for reducing the risk or progression of cardiovascular diseases or risk factors thereof in a subject, comprising administering to the subject a pharmaceutical composition in an amount sufficient to reduce non-HDL cholesterol levels and triglyceride levels, the composition containing natural or synthetic omega-3 fatty acids, or pharmaceutically acceptable esters, derivatives, conjugates, precursors or salts thereof, or mixtures thereof, and one or more pharmaceutically acceptable excipients.

58. The method of claim 57, wherein the omega-3 fatty acids comprise at least 60% by weight of EPA and DHA as compared to the total fatty acid content of the composition, and wherein the EPA and DHA are in a weight ratio of EPA:DHA of from 99:1 to 1:99.

59. The method of claim 57, wherein the omega-3 fatty acids are present in a concentration of at least 70% by weight as compared to the total fatty acid content of the composition.

60. The method of claim 58, wherein the EPA and DHA are in a weight ratio of EPA:DHA of from 1:2 to 2:1.

61. The method of claim 57, wherein the subject has a baseline triglyceride level of greater than or equal to 500 mg/dL.

62. The method of claim 57, wherein the subject has a baseline triglyceride level between 200 mg/dL to 499 mg/dL.
